(19) United States
(12) Patent Application Publication
(10) Pub. No.: US 2026/0272530 A1
(43) Pub. Date: Sep. 17, 2026

(54) TEXTURED DRIVER TIP DEVICE AND METHODS

(71) Applicant: Acumed LLC, Hillsboro, OR (US)

(72) Inventors: Brayden Reiland, Portland, OR (US); Travis Sewell, Portland, OR (US); Steve Crawford, Portland, OR (US)

(21) Appl. No.: 19/564,849

(22) Filed: Mar. 12, 2026

Related U.S. Application Data
(60) Provisional application No. 63/771,773, filed on Mar. 14, 2025.

(51) Int. Cl. A61B 17/88; A61B 17/17; A61B 17/56

(52) U.S. Cl. CPC A61B 17/888; A61B 17/17; A61B 2017/564

(57) ABSTRACT

A driver has improved “stick fit” with a head of a bone screw. The driver may include a tip portion defining a central axis and may include an external surface. At least a portion of the external surface may be symmetrical about the central axis. The tip portion may include at least one ridge extending from the external surface. The at least one ridge may provide an interference fit with an internal recess of a screw.

### INCORPORATION BY REFERENCE TO ANY PRIORITY APPLICATIONS

The application claims benefit under 35 U.S.C. § 119(e) to U.S. Provisional Patent Application Ser. No. 63/771,773, filed Mar. 14, 2025, entitled “TEXTURED DRIVER TIP DEVICE AND METHODS”, the disclosure of which is hereby incorporated by reference and made part of this specification as if set forth fully herein in its entirety. Any and all applications for which a foreign or domestic priority claim is identified in the Application Data Sheet as filed with the present application are hereby incorporated by reference under 37 CFR 1.57.

### BACKGROUND

Numerous medical procedures require mating medical instruments with holes or recesses of implants or fixation devices. For example, a drill guide may be mated to a hole in a bone plate to provide a surgeon with proper alignment for drilling through the hole. Similarly, a surgeon may mate a targeting guide within an aperture of a bone plate to aid in proper positioning of a screw. Quick and easy securement of the instrument with the device may be desired.

Another application of mating instruments within a recess includes the use of a driver with a bone screw. Current approaches utilize smooth mating surfaces to establish a stick-fit interface between the driver and the screw. However, the current reliance on smooth surface interactions often results in suboptimal, inconsistent performance. One recurring issue in clinical practice is the difficulty in achieving a reliable stick-fit between the driver and screw.

### SUMMARY

Existing tools and techniques fail to provide consistent stick-fit performance while still being able to easily removed. There remains a need for a solution that enhances the interface between medical instruments and implants, such as drivers and screws.

The present disclosure provides a new and innovative application of an uneven surface finish to an external mating surface of a medical instrument to improve an interference fit between the medical instrument and a recess or hole. In some embodiments, the present disclosure provides mating instruments including an uneven surface finish to an external mating surface of a medical instrument. The uneven surface finish can improve an interference fit between the medical instrument and a recess or hole. In some implementations, the present disclosure provides a driver with an uneven surface finish and methods for screw insertion into a bone using the driver. The driver provides enhanced stick-fit performance with bone screws. For example, by creating an uneven surface finish, the driver establishes an interference fit with the screw, ensuring reliable engagement between the screw and the driver. In the present disclosure, the uneven surface finish includes intentionally created structures with predetermined geometry and resulting in an uneven surface. The intentionally created structures can include, e.g. but not limited to, ridges, recesses, and the like. The uneven surface finish in the context of the present disclosure does not include random surface unevenness of a coarse surface.

In some implementations, a driver for bone screw insertion is provided. The driver may include a tip portion defining a central axis and including an external surface. At least a portion of the external surface may be symmetrical about the central axis. The tip portion may include at least one ridge extending from the external surface. In some embodiments, the at least one ridge is substantially perpendicular to the central axis.

In some implementations, a method of screw insertion using the driver disclosed herein is provided. The method may include aligning the driver against a recess of the bone screw and inserting the tip portion into the recess of the bone screw. The at least one ridge may create multiple contact points with internal features of the bone screw to establish an interference fit between the driver and the bone screw. The method may further include positioning the bone screw at an entry point on a bone surface, and applying rotational force to the driver.

In some aspects, the techniques described herein relate to a medical instrument configured to be mated with an implant or fixation device, the instrument including: a mating portion configured to mate with the implant or fixation device, the mating portion defining a central axis and including an external surface extending circumferentially around the central axis, wherein at least a portion of the external surface can be symmetrical about the central axis, and wherein the mating portion can further include at least one ridge contiguous with the external surface and extending from the external surface in a direction transverse to the central axis.

In some aspects, the techniques described herein relate to an instrument, wherein the at least one ridge can continuously and circumferentially extend around the external surface of the mating portion.

In some aspects, the techniques described herein relate to an instrument, wherein the at least one ridge can include a plurality of ridges that each extend from the external surface to a same predefined height.

In some aspects, the techniques described herein relate to an instrument, wherein the at least one ridge can include a plurality of ridges that extend from the external surface to a predefined height, wherein the predefined height of at least some of the plurality of ridges can be different.

In some aspects, the techniques described herein relate to an instrument, wherein the predefined height of the plurality of ridges can increase from a first end of the mating portion to a second end of the mating portion along the central axis.

In some aspects, the techniques described herein relate to an instrument, wherein the at least one ridge can include one or more discontinuous sections around the external surface.

In some aspects, the techniques described herein relate to an instrument, wherein the at least one ridge can include a geometry that, in a side view, includes a ramp up portion and a ramp down portion, and wherein the ramp up portion and the ramp down portion can meet to form a peak.

In some aspects, the techniques described herein relate to an instrument, wherein the peak can be angled towards one of the ramp up portion or the ramp down portion to form a barb.

In some aspects, the techniques described herein relate to an instrument, wherein the at least one ridge can include a geometry that, in a side view, includes a ramp up portion, a ramp down portion, and a step portion between the ramp up portion and the ramp down portion.

In some aspects, the techniques described herein relate to an instrument, wherein the ramp up portion and the ramp down portion can be asymmetrical.

In some aspects, the techniques described herein relate to an instrument, wherein the external surface of the mating portion can include one or more grooves positioned parallel to the central axis.

In some aspects, the techniques described herein relate to an instrument, wherein the mating portion can include a hexalobe pattern, wherein the at least one ridge can be positioned along the hexalobe pattern and transverse to the hexalobe pattern.

In some aspects, the techniques described herein relate to an instrument, wherein the at least one ridge can include a helical structure around the mating portion.

In some aspects, the techniques described herein relate to an instrument, wherein the helical structure can include a single-start thread or a multi-start thread.

In some aspects, the techniques described herein relate to an instrument, wherein a pitch of the helical structure can be variable along the central axis.

In some aspects, the techniques described herein relate to an instrument, wherein the at least one ridge can extend outward from the external surface of the mating portion.

In some aspects, the techniques described herein relate to an instrument, wherein the at least one ridge can extend inward from the external surface of the mating portion.

In some aspects, the techniques described herein relate to an instrument, wherein the instrument can be a driver and the implant can be a screw, the driver further including an attachment mechanism configured to secure the driver to an external device.

In some aspects, the techniques described herein relate to an instrument, wherein the instrument can be a drill guide or a targeting guide, the mating portion of the drill guide or the targeting guide configured to engage an aperture in a bone plate.

In some aspects, the techniques described herein relate to a method of screw insertion using a driver, wherein the driver can include a tip portion configured to mate with a bone screw, the tip portion defining a central axis and including an external surface, wherein the tip portion can further include at least one ridge extending from and contiguous with the external surface and wherein the at least one ridge can be transverse to the central axis, the method including: aligning the driver against a recess of the bone screw; inserting the tip portion into the recess of the bone screw by applying a predetermined axial force, wherein the at least one ridge can create multiple contact points with internal features of the recess of the bone screw to establish an interference fit between the driver and the bone screw; positioning the bone screw at an entry point on a bone surface; and applying a rotational force to the driver.

In some aspects, the techniques described herein relate to a method, wherein inserting the tip portion into the recess of the bone screw can cause a deformation of the recess of the bone screw.

In some aspects, the techniques described herein relate to a method, wherein the at least one ridge can extend 360 degrees around the external surface of the bone screw, and wherein 360 degrees of the at least one ridge can contact the recess of the bone screw.

In some aspects, the techniques described herein relate to a method, wherein the interference fit between the external surface of the driver and the internal features of the bone screw can be established by frictional engagement.

Additional features and advantages of the disclosed methods are described in, and will be apparent from, the following Detailed Description and the Figures.

### BRIEF DESCRIPTION OF THE FIGURES

FIG. 1 illustrates an example driver according to the present disclosure.

FIG. 1A illustrates a cross-sectional view of the example driver of the present disclosure coupled with a screw.

FIG. 1B illustrates a cross-sectional view of a standard tapered or “stick fit” driver coupled with a screw.

FIG. 2A illustrates a tip portion of the driver of FIG. 1 including an uneven exterior surface finish according to one embodiment of the present disclosure.

FIG. 2B illustrates a tip portion of the driver of FIG. 1 including an uneven exterior surface finish according to another embodiment of the present disclosure.

FIG. 3 illustrates a magnified view of ridges of a tip portion of a driver according one embodiment of the present disclosure.

FIG. 4 illustrates a magnified view of ridges of a tip portion of a driver according another embodiment of the present disclosure.

FIG. 5 illustrates a tip portion of a driver according to another example of the present disclosure.

FIG. 6A illustrates a drill guide according to an example of the present disclosure.

FIG. 6B illustrates a detailed view of a mating portion of the drill guide of FIG. 6A.

FIG. 7 illustrates a cross-sectional view of the mating portion of the drill guide of FIG. 6A mated with a hole of a bone plate.

FIG. 8 illustrates an assembly of a targeting guide and a bone plate with a plurality of holes according to some embodiments of the present disclosure.

FIG. 9 illustrates a first side (e.g., bottom side) of the targeting guide of FIG. 8.

FIG. 10A illustrates a partial view of a second side (e.g., a top side) of the bone plate of FIG. 8.

FIG. 10B illustrates a detailed view of a hole or recess of the bone plate of FIG. 8 with one or more ridges.

### DETAILED DESCRIPTION OF EXAMPLES

The present disclosure provides a new and innovative application of an uneven surface finish to an external mating surface of a medical instrument or an internal mating surface of a recess or hole to improve an interference fit between the medical instrument and a recess or hole. In some embodiments, the present disclosure provides a medical instrument with an uneven surface finish to an external mating surface or an internal mating surface of a recess or hole configured to receive the instrument to improve an interference fit between the medical instrument and the recess or hole. In some embodiments, the recess or hole is a part of an implant or a fixation device. In some embodiments, the medical instrument is a driver, and the recess is a part of a bone screw.

A medical instrument may need to couple to an implant, for example but not limited to a screw, a plate, etc. The medical instrument may include a tip that can be inserted into an opening, recess, aperture, etc. of the implant. A tip with a constant outer diameter (also referred to as a straight tip) may create what is commonly called a “non-stick fit” with the implant, whereas a tip with a taper (e.g., with a decreasing outer diameter toward the free end of the tip), also referred to as a tapered tip, may create what is commonly called a “stick fit” with the implant. “Stick fit” may refer to that ability of a medical instrument to remain stuck within or engaged with a recess after insertion of the medical instrument into the recess.

In some embodiments of the present disclosure, interference between the external surface of the medical instrument and the hole or recess creates a “stick fit” between the medical instrument and the hole or recess (e.g., without having a tapered tip portion). Such medical instruments may include a straight tip with uneven surface finish. In other embodiments, the stick fit can be created between a tapered tip portion including uneven surface finish and the hole or recess. Insertion of the medical instrument into the recess may include applying an axial force to the instrument. The medical instrument may remain “stuck” within the hole or recess until a force is applied to overcome the retention force. In some embodiments, the retention force is between 5N and 15N, between 15N and 35N, or between 35N and 50N, for example between 20N to 30N. In some embodiments, the retention force may be between about 5N and about 50N, or between about 15N and about 50N, or between about 35N and about 50N.

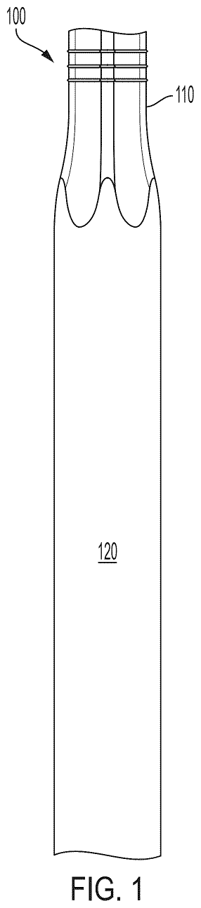

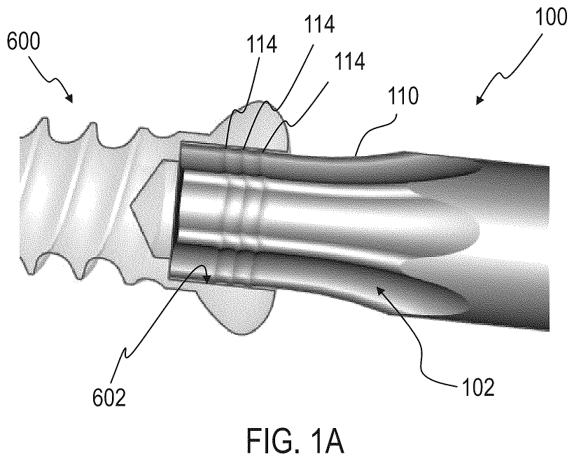

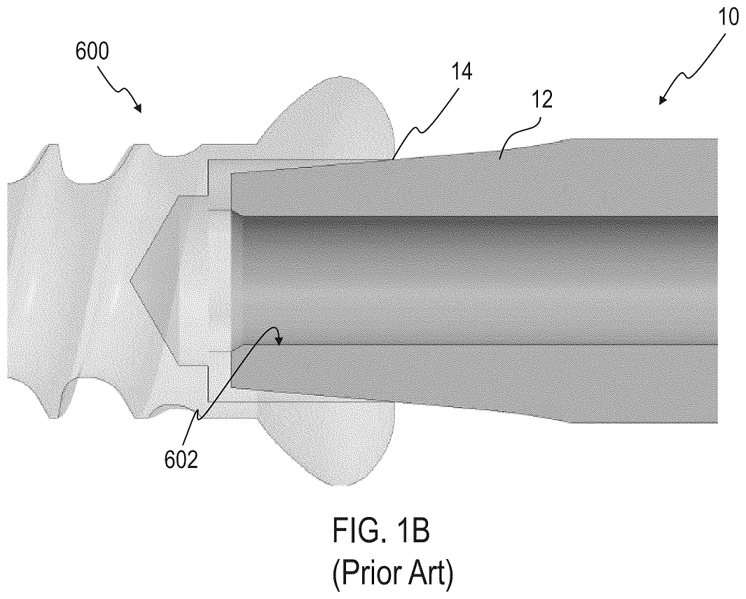

FIG. 1 illustrates an example of a driver 100 according to the present disclosure. The driver 100 provides interference between an internal recess of a bone screw creating a “stick-fit” between the driver 100 and the screw. As shown in FIG. 1, the driver 100 may include a tip portion 110 and a shaft portion 120. The tip portion 110 of the driver 100 may be configured to engage with an internal recess of a screw and therefore the tip portion 110 can also be referred to herein as a mating portion. While FIG. 1 depicts the tip portion 110 with a hexalobe pattern, it can be appreciated that other driver geometries may be used (e.g. hex, cruciform, Phillips head, flat head, dog bone, or any other suitable driver geometries).

The shaft portion 120 of the driver 100 may be configured to apply a torque to the driver 100 and/or the screw. In some embodiments, the shaft portion 120 may include a handle to be grasped by a user (e.g. a surgeon). The handle or grip portion of the driver 100 may be designed to provide an ergonomic and secure hold. The handle may be contoured to fit comfortably in a surgeon's hands, reducing fatigue during extended procedures. In some embodiments, the handle may also include textured areas or grips to further enhance stability and control during screw insertion.

In some embodiments, the shaft portion 120 may include an attachment mechanism. The attachment mechanism may be designed to connect the driver 100 to an external device, such as a surgical drill or motorized tool. In some embodiments, the attachment mechanism may be configured as an AO connector ensuring compatibility with various tools and systems used in clinical applications. For example, the shaft portion 120 may include a free end with a recessed surface and a groove configured to removably couple to a tool. In other embodiments, the attachment mechanism may be a mini-AO connector, offering a more compact design suite for smaller, more precise surgical procedures. In some embodiments, the attachment mechanism may include a Hudson adapter.

The tip portion 110 and the shaft portion 120 may be integrally formed or may be separate components. In some embodiments, the driver 100 may include interchangeable tip portions 110 with the same shaft portion 120. Stated differently, in some embodiments, the tip portion 110 may be removably coupled to the shaft portion 120. The tip portions 110 may be designed for use with different screw types. For example, the tip portions 110 may be optimized for use with screws of different sizes, materials, or shapes.

In some embodiments, the tip portion 110 and/or the shaft portion 120 may include a cannulation along the length. In some embodiments, the cannulation may extend along a whole length of the tip portion 110 and/or the shaft portion 120. In some embodiments, the cannulation may extend along a partial length of the tip portion 110 and/or the shaft portion 120. Alternatively, the tip portion 110 and/or the shaft portion 120 may be non-cannulated (i.e., may have a solid core).

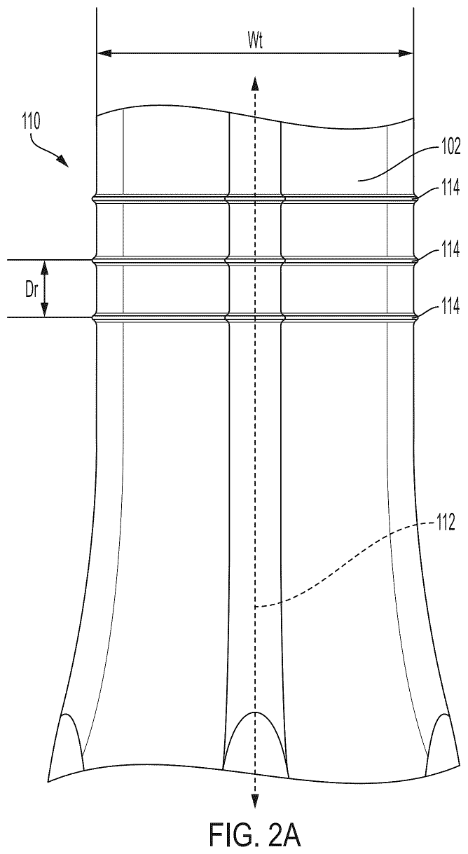

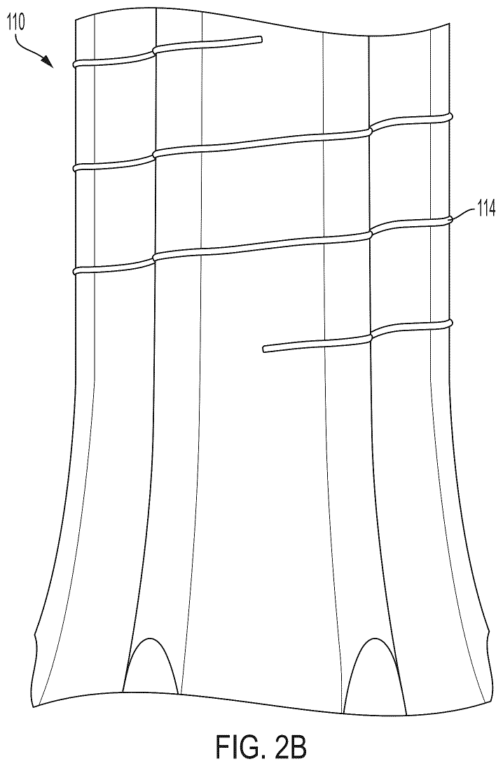

FIGS. 2A and 2B illustrate example tip portions 110 of the driver 100 according to the present disclosure. The tip portion 110 may include an external surface and may define a central axis 112. In other words, the external surface may extend circumferentially around the central axis 112. In some embodiments, at least a portion (or the entirety) of the external surface of the tip portion 110 is symmetrical about the central axis 112. As described previously or elsewhere herein, in some embodiments, the external surface of the tip portion 110 forms a hexalobe pattern as depicted in FIGS. 2A and 2B. For example, in some embodiments, the tip portion 110 includes one or more grooves 102 (e.g. 6 grooves) down a length of the tip portion (e.g. positioned parallel to the length of the tip portion or the central axis 112). In some embodiments, the number of grooves 102 may be based on the driver geometry, such as six grooves for a hexalobe pattern or a different number of grooves for a different driver geometry. However, it can be appreciated that other suitable driver designs may be used (e.g. hex, cruciform, Phillips head, flat head, dog bone). In some embodiments, the driver design is complimentary to the recess of the bone screw.

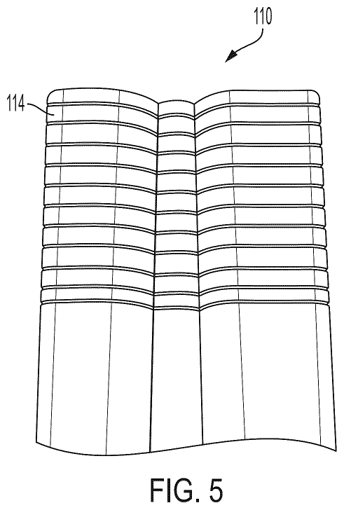

In some embodiments, the external surface of the tip portion 110 has a constant diameter or width Wt along a length of the tip portion 110. In some embodiments, a width Wt or diameter of the external surface of the tip portion 110 (gradually) tapers from a first larger width to a second smaller width (see, e.g., the tip portion as shown in FIG. 5). For example, as shown in FIG. 5, a free end of the tip portion 110 may have the second smaller width while a portion of the tip portion 110 closer to the shaft portion 120 may have the first larger width.

As shown in FIGS. 2A and 2B, the tip portion 110 may include at least one ridge 114 that extends (e.g., outwardly) from the external surface. In some embodiments, the at least one ridge can extend in a direction that is transverse (e.g., perpendicular or slightly offset from perpendicular) to the central axis. In some embodiments, the at least one ridge 114 is substantially perpendicular to the central axis 112 of the tip portion 110. The at least one ridge 114 may serve as a primary mechanism of engagement between the driver 100 and the bone screw, providing an interference fit (e.g., friction fit) between the driver 100 and the screw. In some embodiments, the tip portion 110 may include one ridge 114, two ridges 114, three ridges 114, four ridges 114, five ridges 114, six ridges 114, or any suitable number of ridges extending from the external surface.

In some embodiments, the ridge(s) 114 may extend fully (e.g., 360°) around the external surface of the tip portion 110. The continuous nature of the ridge 114 may create multiple points of contact between the driver 100 and the screw, increasing the interference between the two. In some embodiments, the ridge(s) 114 may extend partially (e.g. noncontinuous or less than 360°) around an external surface of the tip portion 110. In some embodiments, some of the ridges 114 extend fully around the external surface of the tip portion 110 and some of the ridges 114 extend partially around the external surface of the tip portion 110.

In some embodiments, the tip portion 110 may include a ridge 114 with a helical structure around the external surface of the tip portion 114 similar to a thread on a screw, as shown in FIG. 2B. In some embodiments, the helical ridge 114 is a single start thread or a multi-start thread. In some embodiments, the helical ridge 114 rotates clockwise or counterclockwise around the tip portion 110. In some embodiments, the pitch of the helical structure (either single start or multi-start) is variable or the same along the length of the tip portion 110.

As shown in FIG. 2A, in some embodiments, the external surface of the tip portion 110 may feature (e.g., include) multiple ridges 114 arranged at varying positions along the length of the tip portion 110. In some embodiments, the ridges 114 may be equally spaced. Additionally or alternatively, the spacing between the ridges 114 may vary. In some embodiments, the distance Dr between the ridges may be between 0.010 inches and 0.015 inches. In some embodiments, Dr between the ridges may be greater than the length of the ridge in the longitudinal direction. In some embodiments, the distance between the ridges may impact the retention force of the driver within the screw.

In other words, the tip portion of the medial instrument disclosed herein can include tapering on either or both of the height of the ridges (or other uneven surface finish features disclosed herein) and the outer diameter of the tip portion Wt.

The medical instrument with a straight (that is, constant outer diameter) tip portion and ridges (such as disclosed herein and as illustrated in FIGS. 1 and 2A-2B) or any other types of uneven surface finish as disclosed herein can be advantageous over a tapered tip portion that does not include any uneven surface finish at least because of improved torque transmission. As shown in FIG. 1A, the multiple ridges 114 of the tip portion 110 can each contact the inner surface 602 of the recess of the implant 600 (e.g., a screw) for torque transmission. The tip portion 110 can extend deep into the recess of the implant 600 (for example, all the way to the deep end of the recess). In contrast, as shown in FIG. 1B, the tapered tip portion 12 of a standard “stick fit” medical instrument 10 may only contact the inner surface 602 of the implant 600 near the opening of the recess on the implant 600. The contact surface 14 of the tapered tip portion 12 constitutes a ring contact for torque transmission. The tapered tip portion 12 cannot extend as deep into the recess of the 600 due to the tapering of the outer diameter of the tip portion 12. Compared to the standard tapered tip (that is, without an uneven surface finish as disclosed herein), the straight tip portion with the uneven surface finish can improve user experience by providing greater torque transmission.

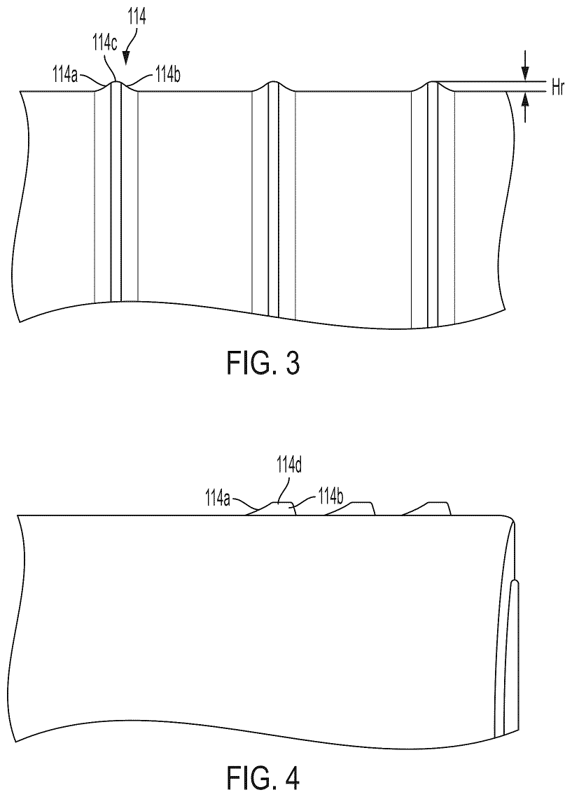

FIGS. 3 and 4 show magnified views of example ridges 114 according to examples of the present disclosure. The ridge 114 may have any of the features of the ridge(s) 114 disclosed herein with reference to FIGS. 2A and 2B. In some embodiments such as more clearly shown in FIGS. 3 and 4, the one or more ridge 114 are contiguous with the external surface, and/or monolithic with the external surface. In some embodiments, the one or more ridges 114 are rigidly formed with the external surface such that the one or more ridges 114 are not compressible or retractable relative to the external surface (unlike, e.g., a spring-biased mechanism).

In some embodiments, the ridge(s) 114 may include a ramp-up portion 114a and a ramp-down portion 114b. In some embodiments, the ramp-up portion 114a and the ramp-down portion 114b may extend (e.g., outwardly) from the external surface. As shown in FIG. 3, the ramp-up portion 114a and the ramp-down portion 114b may meet to form a peak 114c. In some embodiments, the ramp-up portion 114a and the ramp down portion 114b may meet to form a barb, such that the peak is angled toward one of the ramp-up or ramp-down portions.

As shown in FIG. 4, in some embodiments, the ramp-up portion 114a and the ramp-down portion 114b may meet at a step portion 114d. The step portion 114d may be substantially parallel or transverse to the central axis 112 of the tip portion 110. In some embodiments, the ramp-up portion 114a and/or the ramp-down portion 114b may be a flat surface. In some embodiments, the ramp-up portion 114a and/or the ramp-down portion 114b may be a curved surface.

In some embodiments, such as FIG. 3, the ridge(s) 114 may be symmetrical such that the ramp-up portion 114a is a mirror image of or symmetrical with the ramp-down portion 114b. In other embodiments, such as FIG. 4, the ridge(s) may be asymmetrical, with varying angles or profiles. The ramp-up portion 114a and/or the ramp-down portion 114b may extend from the external surface at an angle. In some embodiment, the angle of the ramp-up portion 114a is the same as the angle of the ramp-down portion 114b. In other embodiments, the angle of the ramp-up portion 114a is different than the angle of the ramp-down portion 114b.

In some embodiments, the ridges 114 extend from the external surface to a predefined height Hr. The height Hr of the ridges 114 may be uniform across the length of the tip portion 110. Additionally or alternatively, in some configurations, the height Hr of the ridges 114 may vary along the tip portion 110. In some embodiments, the ridges 114 (gradually) increase in height from one end of the tip portion 110 to the other. In some embodiments, the height Hr of each ridge 114 is the same or different. In some embodiments, the predefined height Hr is between 0.001 inches and 0.002 inches. In some embodiments, the height Hr of the ridge 114 may affect the friction force between the driver 100 and the bone screw. For example, a higher ridge height may result in a higher friction force and a smaller ridge height may result in a lower friction force.

FIG. 5 illustrates a tip portion 110 according to another example of the present disclosure. As noted elsewhere in the present disclosure, the tip portion 110 as illustrated in FIG. 5 can be tapered, e.g., with the outer diameter of the tip portion 110 decreasing towards the free end of the tip portion. As shown in FIG. 5, in some embodiments, the at least one ridge 114 as disclosed herein may extend inward from the external surface of the tip portion 110. The ridge(s) 114 may create a textured finish on the external surface of the tip portion 110 to enhance the friction between the driver 100 and the screw recess. In some embodiments, the tip portion 110 may include at least one ridge 114 extending outwardly form the external surface of the tip portion 110 and at least one ridge 114 extending inwardly from the external surface of the tip portion 110.

The present disclosure provides a method of bone screw insertion using the driver 100 disclosed herein. The method may include aligning the driver 100, such as the tip portion 110 of the driver 100, against a recess of a bone screw and inserting the tip portion 110 into the recess of the bone screw. In some embodiments, inserting the tip portion 110 into the recess of the bone screw includes applying an axial force to the driver 100 in the direction of the bone screw.

In some embodiments, the shape of the tip portion 110 of the driver 100 is complimentary to the shape of the recess of the bone screw. The ridge(s) 114 of the tip portion 110 may create multiple points of contact with the internal features of the bone screw to establish an interference fit (“stick fit”) between the driver 100 and the bone screw. In some embodiments, 360° of the driver, such as the ridge 114, contacts the screw recess after insertion and during the “stick fit”. In some embodiments, the width of the tip portion 110 of the driver 100 at the ridge(s) 114 is greater than the width of the internal recess of the screw.

In some embodiments, the material of the tip portion 110 of the driver 100 is harder than the material of the screw such that the tip portion 110 (e.g., the ridge(s) 114) form micro-deformations in the drive socket of the screw. For example, in some embodiments, the screw is made of a titanium alloy or cobalt chrome, and the driver 100 tip portion 110 is made of 420 MOD (X15TN) or similar.

In some embodiments, a surgeon may couple the driver 100 to the bone screw via the interference fit between the ridge(s) 114 and the internal recess of the bone screw and move the driver 100 and bone screw in unison to the surface of the bone. In some embodiments, a surgeon may couple the driver 100 to a recess of a bone plate to transfer the bone plate to the surface of the bone. The surgeon may use the interaction between the bone plate and the driver to properly position the bone plate on the bone. This may prevent the surgeon from having to hold the bone screw or bone plate in one hand and the driver in the other hand.

In some embodiments, the method may include positioning the bone screw at an entry point on the bone surface. Further, the method may include applying a rotational force to the driver 100 to insert the screw into the bone. In some embodiments, the driver 100 and the bone screw remain engaged by the friction fit or “being stuck” throughout the method. After the bone screw is inserted into the bone, the method may include removing the driver 100 from the screw. In some embodiments, the force required to remove the driver 100 from the screw may be between about 15N and about 35N, or any amount of force (e.g., retention force) disclosed herein.

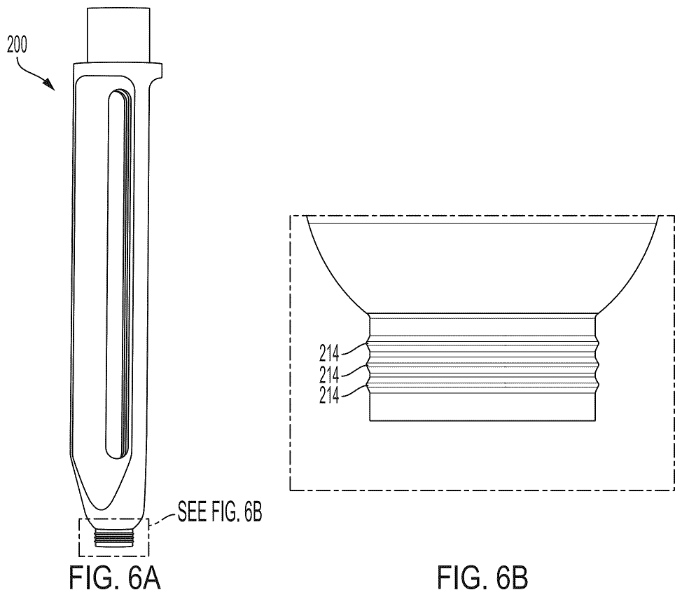

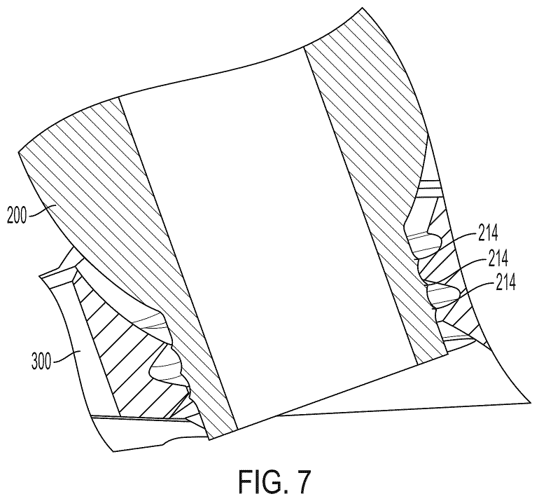

As discussed previously, it can be appreciated that the uneven surface finish (e.g., including at least one ridge) may be applied to the external surface of other mating features of medical instruments to create an interference fit with a recess of an implant or fixation device. In some embodiments, the medical instrument may be a drill guide, and the hole or recess may be within a bone plate. FIGS. 6A, 6B, and 7 illustrate such a drill guide 200 according to an example of the present disclosure.

As illustrated in FIGS. 6A, 6B, and 7, a mating portion of the drill guide 200 may include one or more ridges 214 configured to create an interference fit with an internal recess of an implant, such as a bone plate (see, e.g., bone plate 300 in FIG. 7). The ridge(s) 214 may be the same as and/or may incorporate any of the features of ridge(s) 114 described herein in relation to driver 100. The drill guide 200 may include an internal cannulation configured to receive a drill.

As shown in FIG. 7, the ridge(s) 214 of the drill guide 200 may engage with an internal surface of a hole in the bone plate 300. While FIG. 7 illustrates a threaded hole in the bone plate 300 and the ridge(s) 214 interacting with the minor diameter in the hole, in some embodiments, the hole is non-threaded. The drill guide 200 is configured to remain “stuck” within the hole of the bone plate after application of an axial force to insert the drill guide 200 into the hole. In some embodiments, the drill guide 200 remains within the hole until a predefined force is used to remove the drill guide from the plate, overcoming the friction force between the ridge(s) 214 and the hole.

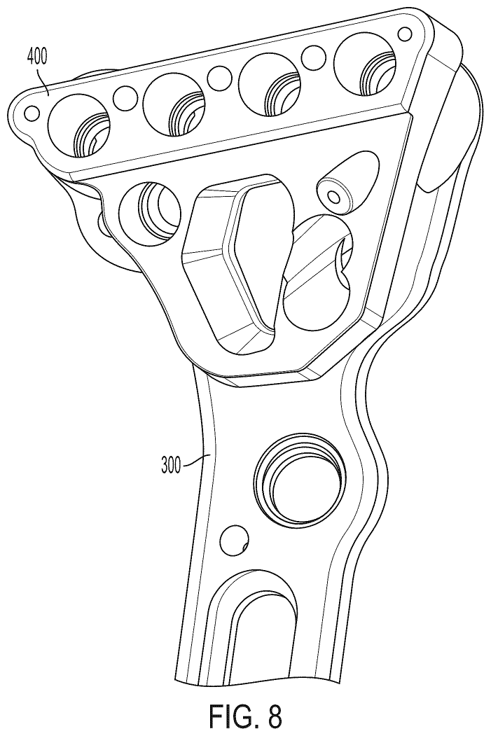

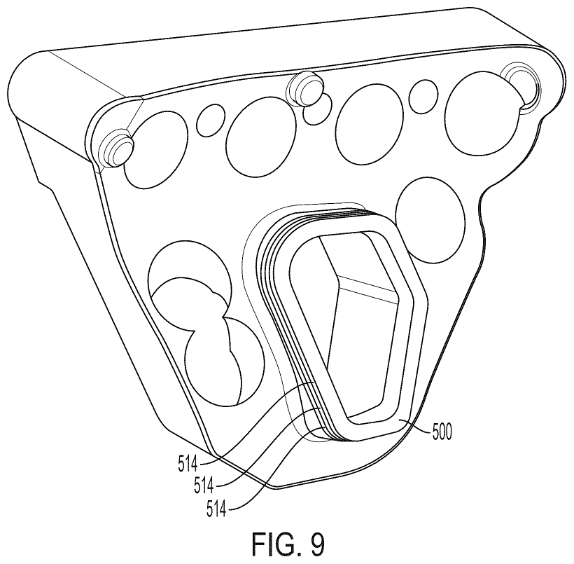

In some embodiments, the medical instrument may include a targeting guide 400 and the hole or recess (or an aperture or any other suitable mating feature) may be within a bone plate 300 as illustrated in FIG. 8. FIG. 8 illustrates an example targeting guide 400 and a bone plate 300. The bone plate 300 can include a plurality of holes or recesses (or an aperture or any other suitable mating feature). The holes or recesses of the bone plate 300 may align with the holes or recesses of the targeting guide 400. The targeting guide 400 may include a post 500 with one or more ridges 514 as described herein configured to create a “stick fit” between the targeting guide 400 and the bone plate 300. The one or more ridges 514 may include any of the features of the ridges 114 as described herein. As shown in FIG. 9, the post 500 may extend from a bottom surface of the targeting guide 400. The bottom surface of the targeting guide 400 can be the surface that contacts a top surface of the bone plate 300. For example, in some embodiments, the post 500 may be integral with the targeting guide 400 and may be sized and shaped to fit into a hole or recess (e.g., a corresponding hole or recess) within the bone plate (e.g., bone plate 300). In some embodiments, the post may be a separate component, configured to be inserted into a hole in the targeting guide 400 and a hole in the bone plate 300.

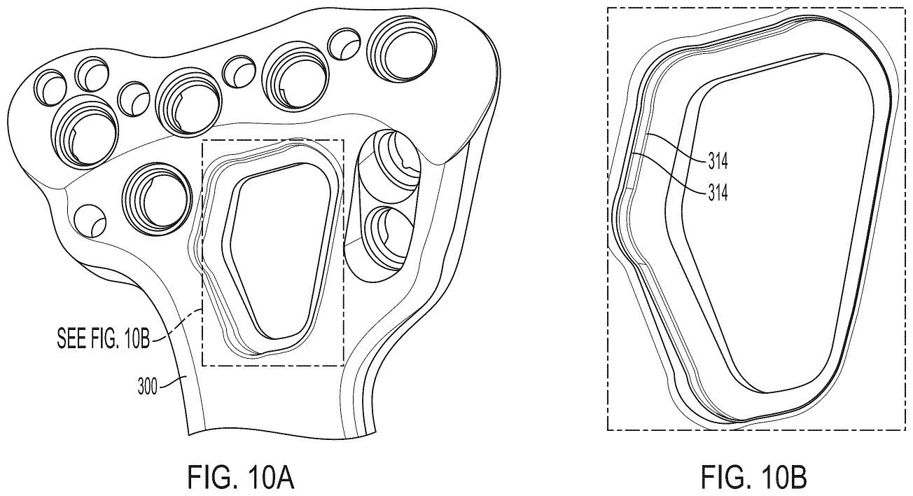

As depicted in FIGS. 10A and 10B, in some embodiments, the one or more ridges 314 as disclosed herein may be located within the hole or recess of the bone plate 300. The one or more ridges 314 may include any of the features of the ridges 114 as described herein. The one or more ridges 314 may be in addition to or alternative to the one or more ridge 514 on the post of the targeting guide 400.

Without further elaboration, it is believed that one skilled in the art can (use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

### Additional Embodiments

Various aspects of the subject matter described herein are also set out in the following additional embodiments, and one or more features of the following embodiments can be incorporated in any other embodiments disclosed herein:

Embodiment 1. A driver for screw insertion comprising: a tip portion defining a central axis and comprising an external surface, wherein at least a portion of the external surface is symmetrical about the central axis, and wherein the tip portion further comprises at least one ridge extending from the external surface and wherein the at least one ridge is substantially perpendicular to the central axis.

Embodiment 2. The driver of embodiment 1, wherein the at least one ridge continuously extends around the external surface of the tip portion.

Embodiment 3. The driver of any one of embodiments 1-2, wherein the at least one ridge comprises a peak spaced at a position between 0 degrees and 360 degrees around the tip portion.

Embodiment 4. The driver of any one of embodiments 1-3, wherein the at least one ridge extends from the external surface to a predefined height.

Embodiment 5. The driver of embodiment 4, wherein the at least one ridge comprises a plurality of ridges, wherein the predefined height of each of the plurality of ridges is the same.

Embodiment 6. The driver of embodiment 4, wherein the at least one ridge comprises a plurality of ridges, wherein the predefined height of each of the plurality of ridges is different.

Embodiment 7. The driver of embodiment 6, wherein the predefined height of the plurality of ridges increases from a first end of the driver to a second end of the driver.

Embodiment 8. The driver of any one of embodiments 1-7, wherein the at least one ridge comprises one or more discontinuous sections around the external surface.

Embodiment 9. The driver of any one of embodiments 1-8, wherein the at least one ridge comprises a ramp up portion and a ramp down portion, and wherein the ramp up portion and the ramp down portion meet to form a peak.

Embodiment 10. The driver of embodiment 9, wherein the peak is angled towards one of the ramp up portion or the ramp down portion to form a barb.

Embodiment 11. The driver of any one of embodiments 1-10, wherein the at least one ridge comprises a ramp up portion, a ramp down portion, and a step portion between the ramp up portion and the ramp down portion.

Embodiment 12. The driver of embodiment 11, wherein the ramp up portion and the ramp down portion are asymmetrical.

Embodiment 13. The driver of embodiment 11, wherein the step portion is substantially parallel to the central axis.

Embodiment 14. The driver of any one of embodiments 1-13, wherein the at least one ridge comprises a plurality of ridges, wherein the plurality of ridges are spaced equidistantly along the length of the tip portion.

Embodiment 15. The driver of any one of embodiments 1-14, wherein the at least one ridge comprises a plurality of ridges, wherein a distance between each of the plurality of ridges is different.

Embodiment 16. The driver of any one of embodiments 1-15, wherein the external surface of the tip portion comprises one or more grooves positioned parallel to a length of the tip portion to form a hexalobe pattern.

Embodiment 17. The driver of embodiment 16, wherein the at least one ridge is positioned along the hexalobe pattern and substantially perpendicular to the hexalobe pattern.

Embodiment 18. The driver of any one of embodiments 1-17, wherein the at least one ridge comprises a plurality of ridges, wherein the plurality of ridges comprises between two to six ridges.

Embodiment 19. The driver of any one of embodiments 1-18, wherein a width of the tip portion excluding the at least one ridge is constant along the central axis.

Embodiment 20. The driver of any one of embodiments 1-19, wherein a width of the tip portion, excluding the at least one ridge, tapers along the central axis.

Embodiment 21. The driver of any one of embodiments 1-20, wherein the at least one ridge comprises a helical structure around the tip portion.

Embodiment 22. The driver of embodiment 20, wherein the helical structure comprises a single-start thread or a multi-start thread.

Embodiment 23. The driver of embodiment 21, wherein the single-start thread or the multi-start thread rotates in a clockwise or a counterclockwise direction.

Embodiment 24. The driver of embodiment 21, wherein pitch of the single-start thread or the multi-start thread is variable along the length of the tip portion.

Embodiment 25. The driver of any one of embodiments 1-24, wherein the at least one ridge extends outward from the external surface of the tip portion.

Embodiment 26. The driver of any one of embodiments 1-25, wherein the at least one ridge extends inward from the external surface of the tip portion.

Embodiment 27. The driver of any one of embodiments 1-26, wherein the driver comprises a solid core.

Embodiment 28. The driver of any one of embodiments 1-27, wherein the driver is cannulated.

Embodiment 29. The driver of any one of embodiments 1-28, further comprising an attachment mechanism configured to secure the driver to an external device, wherein the attachment mechanism is an AO connector.

Embodiment 30. The driver of embodiment 29, wherein the attachment mechanism is a mini-AO connector.

Embodiment 31. The driver of embodiment 29, wherein the attachment mechanism is a Hudson adapter.

Embodiment 32. A method of screw insertion using a driver, wherein the driver comprises a tip portion defining a central axis and comprising an external surface, wherein at least a portion of the external surface is symmetrical about the central axis, wherein the tip portion further comprises at least one ridge extending from the external surface and wherein the at least one ridge is substantially perpendicular to the central axis, the method comprising: aligning the driver against a recess of the bone screw; inserting the tip portion into the recess of a bone screw, wherein the at least one ridge creates multiple contact points with internal features of the bone screw to establish an interference fit between the driver and the bone screw; positioning the bone screw at an entry point on a bone surface; and applying rotational force to the driver.

Embodiment 33. The method of embodiment 32, wherein inserting the tip portion into the recess of the bone screw causes a deformation into the recess of the bone screw.

Embodiment 34. The method of any one of embodiments 32-33, wherein the at least one ridge extends 360 degrees around the external surface of the bone screw, and wherein 360 degrees of the at least one ridge contacts the recess of the bone screw.

Embodiment 35. The method of any one of embodiments 32-34 wherein the interference fit between the external surface of the driver and the internal features of the bone screw is established by frictional engagement.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” “some cases,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” “some cases,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

When the position relation between two parts is described using the terms such as “on,” “above,” “below,” “under,” and “next,” one or more parts may be positioned between the two parts unless the terms are used with the term “immediately” or “directly.” Similarly, as used herein, the terms “attachable,” “attached,” “connectable,” “connected,” or any similar terms may include directly or indirectly attachable, directly or indirectly attached, directly or indirectly connectable, and directly or indirectly connected.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “at least one of X or Y” or “at least one of X and Y” should be interpreted as X, or Y, or X and Y.

Additionally, in describing the components of the system of the present disclosure, there may be terms used like first, second, third, and fourth. These terms may be used for the purpose of differentiating one component from the other, but not to imply or suggest the substances, order, sequence, or number of the components.

It should be understood that various changes and modifications to the examples described herein will be apparent to those skilled in the art. Such changes and modifications can be made without departing from the spirit and scope of the present subject matter and without diminishing its intended advantages. It is therefore intended that such changes and modifications be covered by the appended claims.

What is claimed is:

1. A medical instrument configured to be mated with an implant or fixation device, the instrument comprising:
a mating portion configured to mate with the implant or fixation device, the mating portion defining a central axis and comprising an external surface extending circumferentially around the central axis,
wherein at least a portion of the external surface is symmetrical about the central axis, and
wherein the mating portion further comprises at least one ridge contiguous with the external surface and extending from the external surface in a direction transverse to the central axis.

2. The instrument of claim 1, wherein the at least one ridge continuously and circumferentially extends around the external surface of the mating portion.

3. The instrument of claim 1, wherein the at least one ridge comprises a plurality of ridges that each extend from the external surface to a same predefined height.

4. The instrument of claim 1, wherein the at least one ridge comprises a plurality of ridges that extend from the external surface to a predefined height, wherein the predefined height of at least some of the plurality of ridges are different.

5. The instrument of claim 4, wherein the predefined height of the plurality of ridges increases from a first end of the mating portion to a second end of the mating portion along the central axis.

6. The instrument of claim 1, wherein the at least one ridge comprises one or more discontinuous sections around the external surface.

7. The instrument of claim 1, wherein the at least one ridge comprises a geometry that, in a side view, includes a ramp up portion and a ramp down portion, and wherein the ramp up portion and the ramp down portion meet to form a peak.

8. The instrument of claim 7, wherein the peak is angled towards one of the ramp up portion or the ramp down portion to form a barb.

9. The instrument of claim 1, wherein the at least one ridge comprises a geometry that, in a side view, includes a ramp up portion, a ramp down portion, and a step portion between the ramp up portion and the ramp down portion.

10. The instrument of claim 9, wherein the ramp up portion and the ramp down portion are asymmetrical.

11. The instrument of claim 1, wherein the external surface of the mating portion comprises one or more grooves positioned parallel to the central axis.

12. The instrument of claim 1, wherein the mating portion includes a hexalobe pattern, wherein the at least one ridge is positioned along the hexalobe pattern and transverse to the hexalobe pattern.

13. The instrument of claim 1, wherein the at least one ridge comprises a helical structure around the mating portion.

14. The instrument of claim 13, wherein the helical structure comprises a single-start thread or a multi-start thread.

15. The instrument of claim 13, wherein a pitch of the helical structure is variable along the central axis.

16. The instrument of claim 1, wherein the at least one ridge extends outward from the external surface of the mating portion.

17. The instrument of claim 1, wherein the at least one ridge extends inward from the external surface of the mating portion.

18. The instrument of claim 1, wherein the instrument is a driver and the implant is a screw, the driver further comprising an attachment mechanism configured to secure the driver to an external device.

19. The instrument of claim 1, wherein the instrument is a drill guide or a targeting guide, the mating portion of the drill guide or the targeting guide configured to engage an aperture in a bone plate.

20. A method of screw insertion using a driver, wherein the driver comprises a tip portion configured to mate with a bone screw, the tip portion defining a central axis and comprising an external surface, wherein the tip portion further comprises at least one ridge extending from and contiguous with the external surface and wherein the at least one ridge is transverse to the central axis, the method comprising:
aligning the driver against a recess of the bone screw;
inserting the tip portion into the recess of the bone screw by applying a predetermined axial force, wherein the at least one ridge creates multiple contact points with internal features of the recess of the bone screw to establish an interference fit between the driver and the bone screw;
positioning the bone screw at an entry point on a bone surface; and
applying a rotational force to the driver.
